(19) United States
(12) Patent Application Publication
(10) Pub. No.: US 2024/0150809 A1
(43) Pub. Date: May 9, 2024

(54) Method of Evaluating the Biofilm Detachment Efficacy of Compositions

(71) Applicants: Colgate-Palmolive Company, New York, NY (US); The Ohio State University, Columbus, OH (US)

(72) Inventors: Carlo DAEP, Brooklyn, NY (US); Paul STOODLEY, Columbus, OH (US); Erin GLOAG, Columbus, OH (US)

(21) Appl. No.: 18/548,719

(22) PCT Filed: Mar. 3, 2022
(86) PCT No.: PCT/US2022/018634

§ 371 Date: Sep. 1, 2023

Related U.S. Application Data
(60) Provisional application No. 63/156,042, filed on Mar. 3, 2021.

(51) Int. Cl. C12Q 1/18; C12Q 1/02

(52) U.S. Cl. CPC C12Q 1/18; C12Q 1/02

(57) ABSTRACT

Disclosed are methods of monitoring the detachment of a biofilm from the surface of an object or rearrangement of the structure of the biofilm in response to shear stress and also methods of identifying compositions having biofilm detachment activity or evaluating the biofilm detachment efficacy of compositions.

### BACKGROUND

Biofilms are communities of microorganisms, encased in an extracellular polymeric slime (EPS). These communities adhere at either surface interfaces or to neighboring microorganisms. Biofilms are responsible for a number of infectious diseases, where these communities are highly recalcitrant to traditional therapies, promoting the persistence of these infections.

Oral diseases are one of the most common pathologies affecting human health. These diseases are typically associated with dental plaque-biofilms, through either build-up of the biofilm or dysbiosis of the microbial community. Dental plaque is perhaps one of the most widely understood biofilms affecting human health. Oral pathologies typically arise due to poor oral hygiene and diet, that lead to dental plaque build-up or dysbiosis of the plaque microbial community. Together these factors can lead to oral diseases including dental caries, gingivitis and periodontitis. Oral hygiene, including combinations of mechanical dental plaque removal and antimicrobial agents in dentifrices, continues to be the most effective method at preventing the development of these pathologies.

Arginine has emerged as a novel therapy to combat dental plaque. This mechanism has been chiefly attributed to the buffering capacity of arginine metabolism by arginolytic organisms, including Streptococcus gordonii. These organisms encode an arginine deiminase system (ADS), which metabolizes arginine, producing ammonia. This in turn neutralizes acid produced by acidogenic organisms, maintaining a neutral pH within the dental plaque-biofilm. Arginine treatment also promotes S. gordonii growth and prevents the out-growth of cariogenic species, including Streptococcus mutans, in mixed species biofilm models. Exogenous arginine treatment can also reduce microbial coaggregation, and alters the EPS biochemical composition, by preventing the out-growth of S. mutans, and subsequently reducing the amount of insoluble glycans produced by this organism. It has been reported that treatment with low concentrations of arginine promotes the growth of S. gordonii biofilms, while high concentrations of the amino acid reduces biofilm biomass, suggesting that arginine treatment can disrupt dental plaque-biofilm, preventing its build-up. Arginine can disrupt dental plaque-biofilms, and maintain plaque homeostasis, making it an ideal therapeutic to combat the development of oral disease. Despite our understanding of the actions of arginine towards dental plaque-biofilms, it is still unclear how or if arginine effects the mechanical integrity of the dental plaque-biofilm.

There is a need for methods to identify oral care compositions having biofilm detachment activity and evaluate the efficacy of biofilm detachment of oral care compositions.

### BRIEF SUMMARY

In one aspect, the invention provides a method of monitoring the detachment of a biofilm from the surface of an object or rearrangement of the structure of the biofilm in response to shear stress; comprising:
a) providing a biofilm-coated object;
b) immersing the biofilm-coated object in a liquid;
c) spinning the biofilm-coated object with increasing angular velocity (ω), wherein the spinning generates shear stress;
d) monitoring torque (T) in step c) to obtain torque-angular velocity curve;
e) optionally, linearizing and transforming the torque-angular velocity curve into slope (torque^{1/2}/angular velocity)-angular velocity curve; and
f) determining one or more parameters selected from angular velocity (ω) at which the first reduction in torque occurs, shear stress (τ) at which the first reduction in torque occurs, biofilm momentum coefficient (C_{B}), area under the curve (AUC) of the torque-angular velocity curve, and a combination thereof.

In another aspect, the invention provides a method of identifying compositions having biofilm detachment activity or evaluating the biofilm detachment efficacy of compositions; comprising:
a) providing a biofilm-coated object;
b) treating the biofilm-coated object with a composition;
c) immersing the biofilm-coated object in a liquid;
d) spinning the biofilm-coated object with increasing angular velocity (ω), wherein the spinning generates shear stress;
e) monitoring torque (T) in step c) to obtain torque-angular velocity curve;
f) optionally, linearizing and transforming the torque-angular velocity curve into slope (torque^{1/2}/angular velocity)-angular velocity curve
g) determining one or more parameters selected from angular velocity (ω) at which the first reduction in torque occurs, shear stress (τ) at which the first reduction in torque occurs, biofilm momentum coefficient (C_{B}), area under the curve (AUC) of the—angular velocity curve, and a combination thereof, and
h) comparing the one or more parameters of the composition with those of a control composition.

In another aspect, the invention provides a method of reducing or removing biofilm, e.g., dental plaque, on teeth, comprising an effective amount of an oral care composition to the oral cavity of a subject in need, wherein the oral care composition is identified by a method of identifying a composition having biofilm detachment activity disclosed in this disclosure.

### BRIEF DESCRIPTION OF THE DRAWINGS

The present invention will become more fully understood from the detailed description and the accompanying drawings.

FIG. 1 shows Experimental design for the adapted spinning-disc rheometry analysis. Biofilm-coated coupons were attached to an adaptor probe on the rheometer using a threaded tap that was printed onto the back of the coupon. This was immersed in a container filled with reverse osmosis water. A gap thickness of 3.5 cm was set between the coupon and the bottom of the container.

FIG. 2 show S. gordonii biofilms before and after analysis. Prior to analysis, 5 day S. gordonii biofilms, grown on the coupons, were treated with either PBS (untreated control) or with 4% arginine. Images depict biofilms before and after rheometry analysis (labeled). Scale bar indicates 5 mm.

FIG. 3 shows small biofilm detachment events. Top panel is a still image depicting small biofilm detachment event. The torque-angular velocity curve (bottom left panel) and transformed linearized analysis (bottom right panel) at each time are shown. White arrow indicates detached biofilm and black arrows indicate the corresponding change in the curve.

FIG. 4 shows large biofilm detachment events. Top panel is a still image depicting large biofilm detachment event. The torque-angular velocity curve (bottom left panel) and transformed linearized analysis (bottom right panel) at each time are shown. White arrow indicates detached biofilm and black arrows indicate the corresponding change in the curve.

FIG. 5A and FIG. 5B show adapted spinning-disc measurements of untreated S. gordonii biofilm (FIG. 5A) and 4% arginine treated S. gordonii biofilm (FIG. 5B). In each panel, blank indicates analysis for coupon alone with no biofilm. 4 biological replicates were performed, with 2-4 biofilms analyzed for each replicate (total N=11).

FIG. 6A and FIG. 6B show transformed linearized analysis of untreated S. gordonii biofilm (FIG. 6A) and 4% arginine treated S. gordonii biofilm (FIG. 6B). In each panel, blank indicates analysis for coupon alone with no biofilm. 4 biological replicates were performed, with 2-4 biofilms analyzed for each replicate (total N=11).

FIG. 7 shows biofilm momentum coefficient (C_{B}) of (i) blank (coupon alone with no biofilm), (ii) untreated and (iii) 4% arginine treated S. gordonii biofilm, determined according to equation 1 at 200-300 rad·s⁻¹. *p-value<0.05, ns indicates no statistical difference.

FIG. 8 shows Area under the curve (AUC) of torque-angular velocity curves of (i) blank (coupon alone with no biofilm), (ii) untreated and (iii) 4% arginine treated S. gordonii biofilm. *p-value<0.05.

FIG. 9 shows shear stress at the moment of the first reduction in torque of (i) blank (coupon alone with no biofilm), (ii) untreated and (iii) 4% arginine treated S. gordonii biofilm. The first reduction in torque indicates initiation of detachment. Shear stress at the first detachment occurs was calculated according to equation 3. *p-value<0.05.

FIG. 10 shows biofilm momentum coefficient (C_{B}) of (i) untreated, (ii) glycine treated, (iii) lysine treated, and (iv) arginine treated S. gordonii biofilm, determined according to equation 1 at 200-300 rad·s⁻¹. *p-value<0.05, ns indicates no statistical difference.

FIG. 11 shows Area under the curve (AUC) of torque-angular velocity curves of (i) untreated, (ii) glycine treated, (iii) lysine treated, and (iv) arginine treated S. gordonii biofilm. *p-value<0.05, ns indicates no statistical difference.

FIG. 12 shows shear stress at the moment of the first reduction in torque of (i) untreated, (ii) glycine treated, (iii) lysine treated, and (iv) arginine treated S. gordonii biofilm. The first reduction in torque indicates initiation of detachment. Shear stress at the first detachment occurs was calculated according to equation 3. *p-value<0.05, ns indicates no statistical difference.

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight. The amounts given are based on the active weight of the material.

Mechanical analysis of biofilms is becoming more widespread in the field. However, there is currently a lack in analyzing biofilm mechanics in the context of biofilm removal. In this invention, the inventors have adapted spinning-disc rheometry from the field of biofouling as a novel methodology to analyze biofilm detachment from surfaces. It has been found that the spinning-disc rheometry assay is highly sensitive at detecting biofilm detachment and possible structural rearrangements with increasing shear forces and that this methodology is also sensitive at detecting mechanical changes to the biofilm architecture that are not visually apparent.

The present invention provides, in an aspect, a method (Method 1.0) of monitoring the detachment of a biofilm from the surface of an object or rearrangement of the structure of the biofilm in response to shear stress; comprising:
a) providing a biofilm-coated object;
b) immersing the biofilm-coated object in a liquid;
c) spinning the biofilm-coated object with increasing angular velocity (ω), wherein the spinning generates shear stress;
d) monitoring torque (T) in step c) to obtain torque-angular velocity curve;
e) optionally, linearizing and transforming the torque-angular velocity curve into slope (torque^{1/2}/angular velocity)-angular velocity curve; and
f) determining one or more parameters selected from angular velocity (ω) at which the first reduction in torque occurs, shear stress (τ) at which the first reduction in torque occurs, biofilm momentum coefficient (C_{B}), area under the curve (AUC) of the torque-angular velocity curve, and a combination thereof.

For example, the invention includes:
1.1. Method 1.0, wherein the object is a coupon, e.g., a metallic coupon, a tooth or a hydroxyapatite disk.
1.2. Any of the preceding methods, wherein the object is a coupon.
1.3. Any of the preceding methods, wherein the biofilm is a microbial biofilm.
1.4. Any of the preceding methods, wherein the biofilm is formed on the object by incubating microbial organisms in a culture media containing the object for at least 1 day, e.g., at least 2 days, at least 3 days, at least 4 days, at least 5 days, at least 6 days, at least a week, at least 10 days, at least 2 weeks, from 1 days (2 days, 3 days, 4 days, 5 days, 6 days, a week, 10 days or 2 weeks) to 3 weeks, from 1 days (2 days, 3 days, 4 days, 5 days, 6 days, a week, or 10 days) to 2 weeks, from 1 days (2 days, 3 days, 4 days, 5 days, 6 days or a week) to 10 days, from 1 days (2 days, 3 days, 4 days, 5 days, or 6 days) to 1 week, about 1 day, about 2 days, about 3 days, about 4 days, about 5 days, about 6 days, about a week, about 10 days, about 2 weeks, or about 3 weeks.
1.5. Any of the preceding methods, wherein the biofilm is a bacterial biofilm.
1.6. Any of the preceding methods, wherein biofilm is a biofilm of bacteria selected from S. sanguis, S. mutans, S. gordonii, S. parasangiiis, S. rattus, S. milieri, S. anginosus, S. faecalis, A. naeslundii, A. odonolyticus, L. cellobiosus, L. brevis, L. fermentum, P. gingivalis, T. denticola, and a combination thereof.
1.7. Any of the preceding methods, wherein the biofilm is a biofilm of S. gordonii, optionally wherein the biofilm is grown for about 5 days.
1.8. Any of the preceding methods, wherein in step b), the liquid is water, e.g., reverse osmosis water.
1.9. Any of the preceding methods, wherein in step c), the biofilm-coated object is spun at an angular velocity (ω) of from 0 to 300 rad·s⁻¹, optionally across 360 seconds.
1.10. Any of the preceding methods, wherein in step f), the one or more parameters comprise or is angular velocity (ω) at which the first reduction in torque occurs.
1.11. Any of the preceding methods, wherein in step f), the angular velocity (ω) at which the first reduction in torque occurs represents the angular velocity (ω) at which the first detachment of biofilm from the object occurs.
1.12. Any of the preceding methods, wherein in step f), the one or more parameters comprise or is shear stress (τ) at which the first reduction in torque occurs.
1.13. Any of the preceding methods, wherein in step f), the shear stress (τ) at which the first reduction in torque occurs represents the shear stress (τ) at which the first detachment of biofilm from the object occurs.
1.14. Any of the preceding methods, wherein in step f), the one or more parameters comprise or is biofilm momentum coefficient (C_{B}).
1.15. Any of the preceding methods, wherein the biofilm momentum coefficient (C_{B}) is determined between 200-300 rad·s⁻¹.
1.16. Any of the preceding methods, wherein in step f), the one or more parameters comprise or is area under the curve (AUC) of the torque-angular velocity curve.
1.17. Any of the preceding methods, wherein in step f) the one or more parameters comprise angular velocity (ω) at which the first reduction in torque occurs, shear stress (τ) at which the first reduction in torque occurs, biofilm momentum coefficient (C_{B}), and area under the curve (AUC) of the torque-angular velocity curve.

The present invention, in another aspect, provides a method (Method 2.0) of identifying compositions having biofilm detachment activity or evaluating the biofilm detachment efficacy of compositions; comprising:
i) providing a biofilm-coated object;
j) treating the biofilm-coated object with a composition;
k) immersing the biofilm-coated object in a liquid;
l) spinning the biofilm-coated object with increasing angular velocity (ω), wherein the spinning generates shear stress;
m) monitoring torque (T) in step c) to obtain torque-angular velocity curve;
n) optionally, linearizing and transforming the torque-angular velocity curve into slope (torque^{1/2}/angular velocity)-angular velocity curve
o) determining one or more parameters selected from angular velocity (ω) at which the first reduction in torque occurs, shear stress (τ) at which the first reduction in torque occurs, biofilm momentum coefficient (C_{B}), area under the curve (AUC) of the—angular velocity curve, and a combination thereof, and
p) comparing the one or more parameters of the composition with those of a control composition.

For example, the invention includes:
2.1. Method 2.0, wherein the object is a coupon, e.g., a metallic coupon, a tooth or a hydroxyapatite disk.
2.2. Any of the preceding methods, wherein the object is a coupon.
2.3. Any of the preceding methods, wherein the biofilm is a microbial biofilm.
2.4. Any of the preceding methods, wherein the biofilm is formed on the object by incubating microbial organisms in a culture media containing the object for at least 1 day, e.g., at least 2 days, at least 3 days, at least 4 days, at least 5 days, at least 6 days, at least a week, at least 10 days, at least 2 weeks, from 1 days (2 days, 3 days, 4 days, 5 days, 6 days, a week, 10 days or 2 weeks) to 3 weeks, from 1 days (2 days, 3 days, 4 days, 5 days, 6 days, a week, or 10 days) to 2 weeks, from 1 days (2 days, 3 days, 4 days, 5 days, 6 days or a week) to 10 days, from 1 days (2 days, 3 days, 4 days, 5 days, or 6 days) to 1 week, about 1 day, about 2 days, about 3 days, about 4 days, about 5 days, about 6 days, about a week, about 10 days, about 2 weeks, or about 3 weeks.
2.5. Any of the preceding methods, wherein the biofilm is a bacterial biofilm.
2.6. Any of the preceding methods, wherein biofilm is a biofilm of bacteria selected from S. sanguis, S. mutans, S. gordonii, S. parasangiiis, S. rattus, S. milieri, S. anginosus, S. faecalis, A. naeslundii, A. odonolyticus, L. cellobiosus, L. brevis, L. fermentum, P. gingivalis, T. denticola, and a combination thereof.
2.7. Any of the preceding methods, wherein the biofilm is a biofilm of S. gordonii, optionally wherein the biofilm is grown for about 5 days.
2.8. Any of the preceding methods, wherein in step b), the biofilm-coated object is treated with the composition by placing the biofilm-coated object in the composition for at least 10 seconds, e.g., at least 20 seconds, at least 30 seconds, at least 40 seconds, at least 50 seconds, at least 1 minute, at least 1 minute 30 seconds, at least 2 minutes, at least 3 minutes, at least 4 minutes, at least 5 minutes, from 10 seconds (20 seconds, 30 seconds, 40 seconds, 50 seconds, 1 minute, 1 minute 30 seconds, 2 minutes, 3 minutes, 4 minutes or 5 minutes) to 10 minutes, from 10 seconds (20 seconds, 30 seconds, 40 seconds, 50 seconds, 1 minute, 1 minute 30 seconds, 2 minutes, 3 minutes, 4 minutes or 5 minutes) to 7 minutes, from 10 seconds (20 seconds, 30 seconds, 40 seconds, 50 seconds, 1 minute, 1 minute 30 seconds, 2 minutes, 3 minutes, or 4 minutes) to 5 minutes, about 10 seconds, about 20 seconds, about 30 seconds, about 40 seconds, about 50 seconds, about 1 minute, about 1 minute 30 seconds, about 2 minutes, about 3 minutes, about 4 minutes or about 5 minutes.
2.9. Any of the preceding methods, wherein the composition is an oral care composition, optionally wherein the composition is selected from a toothpaste (dentifrice), a prophylactic paste, a tooth powder, a tooth gel (e.g., whitening gel), a chewing gum, a lozenge, a mouthwash, a paint-on gel, and varnish.
2.10. Any of the preceding methods, wherein the composition is an oral care composition selected from a toothpaste, a gel and a mouthwash.
2.11. Any of the preceding methods, wherein the composition is an oral care composition comprising an anti-microbial agent or a basic amino acid in free or salt form, e.g., arginine in free or salt form.
2.12. Any of the preceding methods, wherein the anti-microbial agent is an anti-bacterial agent, e.g., selected from a source of stannous ion, a source of zinc ion, and cetylpyridinium chloride (CPC).
2.13. Any of the preceding methods, wherein the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof, optionally wherein the stannous ion source is selected from stannous fluoride, stannous chloride and a combination thereof.
2.14. Any of the preceding methods, wherein the zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof, optionally wherein the zinc ion source is selected from zinc citrate, zinc oxide, zinc lactate, zinc chloride and a combination thereof.
2.15. Any of the preceding methods, wherein the composition is an oral care composition comprising arginine in free or salt form and cetylpyridinium chloride (CPC).
2.16. Any of the preceding methods, wherein the composition is an oral care composition comprising arginine in free or salt form and a source of stannous ion or zinc ion.
2.17. Any of the preceding methods, wherein the composition is an oral care composition comprising arginine in free or salt form and a source of stannous ion selected from stannous fluoride, stannous chloride and a combination thereof.
2.18. Any of the preceding methods, wherein the composition is an oral care composition comprising arginine in free or salt form and a source of zinc ion source selected from zinc citrate, zinc oxide, zinc lactate, zinc chloride and a combination thereof, e.g., a combination of zinc citrate and zinc oxide.
2.19. Any of the preceding methods, wherein the composition is an oral care composition comprising arginine in free or salt form, a source of stannous ion and a zinc ion source.
2.20. Any of the preceding methods, wherein the composition is a solution, e.g., water or a buffered solution, e.g., phosphate buffered saline (PBS), containing a compound.
2.21. Any of the preceding methods, wherein the compound is an anti-microbial agent or a basic amino acid in free or salt form, e.g., arginine in free or salt form.
2.22. Any of the preceding methods, wherein in step c), the liquid is water, e.g., reverse osmosis water.
2.23. Any of the preceding methods, wherein in step c), the biofilm-coated object is spun at an angular velocity (ω) of from 0 to 300 rad·s⁻¹, optionally across 360 seconds.
2.24. Any of the preceding methods, wherein in step g), the one or more parameters comprise or is angular velocity (ω) at which the first reduction in torque occurs.
2.25. Any of the preceding methods, wherein in step g), the angular velocity (ω) at which the first reduction in torque occurs represents the angular velocity (ω) at which the first detachment of biofilm from the object occurs.
2.26. Any of the preceding methods, wherein in step g), the one or more parameters comprise or is shear stress (τ) at which the first reduction in torque occurs.
2.27. Any of the preceding methods, wherein in step g), the shear stress (τ) at which the first reduction in torque occurs represents the shear stress (τ) at which the first detachment of biofilm from the object occurs.
2.28. Any of the preceding methods, wherein in step g), the one or more parameters comprise or is biofilm momentum coefficient (C_{B}).
2.29. Any of the preceding methods, wherein the biofilm momentum coefficient (C_{B}) is determined between 200-300 rad·s⁻¹.
2.30. Any of the preceding methods, wherein in step g), the one or more parameters comprise or is area under the curve (AUC) of the torque-angular velocity curve.
2.31. Any of the preceding methods, wherein in step g) the one or more parameters comprise angular velocity (ω) at which the first reduction in torque occurs, shear stress (τ) at which the first reduction in torque occurs, biofilm momentum coefficient (C_{B}), and area under the curve (AUC) of the torque-angular velocity curve.
2.32. Any of the preceding methods, wherein reduction in the one or more parameters compared to the control composition indicates that the compound has biofilm detachment activity.

The present invention relates to a method of monitoring the detachment of a biofilm from the surface of an object or rearrangement of the structure of the biofilm in response to shear stress. In some embodiments, the object is a coupon, e.g., a metallic coupon, a tooth or a hydroxyapatite disk. In certain embodiments, the object is a coupon.

Any microbial organisms may be used to form biofilm on the object. Biofilm may be formed on the object by incubating microbial organisms in a culture media containing the object for at least 1 day, e.g., at least 2 days, at least 3 days, at least 4 days, at least 5 days, at least 6 days, at least a week, at least 10 days, at least 2 weeks, from 1 days (2 days, 3 days, 4 days, 5 days, 6 days, a week, 10 days or 2 weeks) to 3 weeks, from 1 days (2 days, 3 days, 4 days, 5 days, 6 days, a week, or 10 days) to 2 weeks, from 1 days (2 days, 3 days, 4 days, 5 days, 6 days or a week) to 10 days, from 1 days (2 days, 3 days, 4 days, 5 days, or 6 days) to 1 week, about 1 day, about 2 days, about 3 days, about 4 days, about 5 days, about 6 days, about a week, about 10 days, about 2 weeks, or about 3 weeks. In some embodiments, the biofilm is a bacterial biofilm. In some embodiments, the biofilm is a biofilm of bacteria selected from S. sanguis, S. mutans, S. gordonii, S. parasangiiis, S. rattus, S. milieri, S. anginosus, S. faecalis, A. naeslundii, A. odonolyticus, L. cellobiosus, L. brevis, L. fermentum, P. gingivalis, T. denticola, and a combination thereof. In certain embodiments, the biofilm is a biofilm of S. gordonii, optionally wherein the biofilm is grown for about 5 days.

In the present invention, spinning disc rheometry analysis is used to measure changes in biofilm deformation, e.g., detachment of biofilm from the surface of an object or rearrangement of the structure of the biofilm, when exposed to varying shear stress. Shear stress is a force tending to cause deformation of a material by slippage along a plane or planes parallel to the imposed stress. In the present invention, shear stress (τ) is generated by rotating the biofilm-coated object in a liquid. In some embodiment, the liquid is water, e.g., reverse osmosis water. Angular velocity (ω) is a vector measure of rotation rate that refers to how fast an object rotates or revolves relative to another point, i.e., how fast the angular position or orientation of an object changes with time. Higher angular velocity (ω) generates greater shear stress (τ). By spinning the biofilm-coated object with increasing angular velocity, the biofilm-coated object is exposed to increasing shear stress. In some embodiments, the biofilm-coated object is spun at an angular velocity (ω) of from 0 to 300 rad·s⁻¹, optionally across 360 seconds.

In the present invention, the detachment of a biofilm from the surface of an object or rearrangement of the structure of the biofilm in response to shear stress is monitored by monitoring torque (T) in response to increasing angular velocity (ω). Torque may be measured by rheometers known in the art, e.g., Discovery Hybrid Rheometer-2 (HD-2) (TA Instruments). Torque, the rotational equivalent of linear force, is a measurement of resistance to rotary motion. Although torque generally increases as angular velocity increases, torque is influenced by the presence of biofilm and the roughness and deformability of the biofilm. Torque is instantly decreased by the detachment of a biofilm from the surface of an object. Thus, the detachment of a biofilm from the surface of an object or rearrangement of the structure of the biofilm in response to shear stress can be detected by monitoring torque (T) in response to increasing angular velocity (ω). In some embodiments, an instant reduction at an angular velocity indicates the detachment of a biofilm from the surface of the object at the angular velocity.

In order to more clearly observe the change in torque, the torque-angular velocity curve may be linearized and transformed. In some embodiments, the torque-angular velocity curve is linearized and transformed into slope (torque^{1/2}/angular velocity)-angular velocity curve. The torque-angular velocity curve is first linearized by taking the square root of the torque and then the running slope of the linearized data is plotted against the angular velocity.

The mechanical properties of a biofilm, e.g., the detachment of a biofilm from the surface of an object or rearrangement of the structure of the biofilm, can be quantified by determining one of more parameters selected from the angular velocity (ω) at which the first reduction in torque occurs, shear stress (τ) at which the first reduction in torque occurs, biofilm momentum coefficient (C_{B}), area under the curve (AUC) of the torque-angular velocity curve, and a combination thereof.

In some embodiments, angular velocity (ω) at which the first reduction in torque occurs is determined to quantify the mechanical properties of a biofilm. The reduction in torque is associated with biofilm detachment events. Lower angular velocity (ω) at which the first reduction in torque occurs means that the biofilm is more easily detached from the object. This parameter can be used to evaluate the biofilm detachment efficacy of tested compositions.

In some embodiment, shear stress (τ) at which the first reduction in torque occurs is converted to shear stress (τ). The angular velocity (ω) can be converted to the shear stress acting on the outer edge of the coupon (τ), as previously described (Hunsucker et al, 2016, Biofouling, 32, 1209-1221), according to equation 3:

τ=\sqrt{τ_{φ}²+τᵣ²} (3)

where τ_{φ} is the shear stress acting in the circumferential direction and τᵣ is the shear stress acting radially.
The shear stress acting in the circumferential direction is described by equation 4:

$$96 {}_{φ} = \frac{{ω}^{2} \cdot {r}^{2}}{{( 4.96 \cdot {log}_{10} Re - 5.74 )}^{2}} \cdot ρ \qquad ( 4 )$$

where Re is the Reynolds number acting at the outer edge of the coupon described by equation 5:

Re=ω·r²/v (5)

where v is the kinematic viscosity (9×10⁻⁷ m²·s⁻¹).
The shear stress acting in the radial direction is described by equation 6:

τᵣ=α·τ_{φ} (6)

where α is the skewness between the shear stress acting in both directions, and is described by equation 7:

$$Ε = \frac{4.395}{4.96 \cdot {log}_{10} Re - 5.74} - 0.0107 \qquad ( 7 )$$

Lower shear stress (τ) at which the first reduction in torque occurs means that the biofilm is more easily detached from the surface. This parameter can be used to evaluate the biofilm detachment efficacy of tested compositions.

In some embodiments, the biofilm momentum coefficient is determined to quantify the mechanical properties of a biofilm. The biofilm momentum coefficient (C_{B}), also referred to as the momentum or torque coefficient, is determined as previously described (Dennington et al, 2015, Surface Topography: Metrology and Properties, 3, 034004). The adapted spinning-disc rheology measurement is most sensitive at detecting changes in torque at the turbulent regime, between 200-300 rad·s⁻¹. Torque within this range has a linear relationship to ω², where the slope of this line (T^{1/2}/ω) equates to C_{B}·k. Therefore, C_{B} can be defined by equation 1:

$${C}_{B} = \frac{slope}{k} \qquad ( 1 )$$

where k is a constant for the system, defined by:

$$k = \frac{ρ \cdot {r}^{5}}{2} \qquad ( 2 )$$

where ρ is the density of the fluid, in this case water (997 kg/m³) and r is the radius of the object. The biofilm momentum coefficient is a dimensionless unit that is an indication of the drag caused by the biofilm, which in turn is related to the thickness and roughness of biofilm. Therefore, a higher coefficient is associated with more drag on the object, due to increased amount of adhered biofilm. In some embodiments, the biofilm momentum coefficient is determined between 200-300 rad·s⁻¹.

In some embodiments, the Area under the curve (AUC) of torque-angular velocity curve is determined to quantify the mechanical properties of a biofilm. The AUC represents the total torque across the whole analyzed range. Lower AUC means that less work is required for rotation of an object. The AUC can be determined using the analysis function in software known in the art, e.g., GraphPad Prism.

The method of monitoring the detachment of a biofilm from the surface of an object or rearrangement of the structure of the biofilm in response to shear stress can be used to identify compositions having biofilm detachment activity or evaluate the biofilm detachment efficacy of compositions. For this purpose, the biofilm-coated object is treated with compositions to be tested prior to the spinning disc rheometry analysis. The biofilm-coated object may be treated with compositions of interest by placing the biofilm-coated object in the compositions for at least 10 seconds, e.g., at least 20 seconds, at least 30 seconds, at least 40 seconds, at least 50 seconds, at least 1 minute, at least 1 minute 30 seconds, at least 2 minutes, at least 3 minutes, at least 4 minutes, at least 5 minutes, from 10 seconds (20 seconds, 30 seconds, 40 seconds, 50 seconds, 1 minute, 1 minute 30 seconds, 2 minutes, 3 minutes, 4 minutes or 5 minutes) to 10 minutes, from 10 seconds (20 seconds, 30 seconds, 40 seconds, 50 seconds, 1 minute, 1 minute 30 seconds, 2 minutes, 3 minutes, 4 minutes or 5 minutes) to 7 minutes, from 10 seconds (20 seconds, 30 seconds, 40 seconds, 50 seconds, 1 minute, 1 minute 30 seconds, 2 minutes, 3 minutes, or 4 minutes) to 5 minutes, about 10 seconds, about 20 seconds, about 30 seconds, about 40 seconds, about 50 seconds, about 1 minute, about 1 minute 30 seconds, about 2 minutes, about 3 minutes, about 4 minutes or about 5 minutes. By shaking the composition containing the biofilm-coated object gently, e.g., at 150 rpm, the biofilm may be treated with the composition more evenly.

The tested composition may be an oral care composition. Illustrative oral care compositions may include, but are not limited to, a toothpaste (dentifrice), a prophylactic paste, a tooth powder, a tooth gel (e.g., whitening gel), a chewing gum, a lozenge, a mouthwash, a paint-on gel, and varnish. In some embodiments, the oral care composition is a toothpaste, a tooth gel or a mouthwash. If the oral care composition to be tested is not a liquid or the biofilm-coated object cannot be properly placed in the oral care composition, the oral care composition may be dissolved in or diluted with a solution, e.g., water or a buffered solution, e.g., phosphate buffered saline (PBS), and then the biofilm-coated object is placed in the diluted composition. The tested oral care composition may comprise an anti-microbial agent or a basic amino acid in free or salt form, e.g., arginine in free or salt form. The anti-microbial agent may be an anti-bacterial agent, e.g., a source of stannous ion, a source of zinc ion, and cetylpyridinium chloride (CPC). The stannous ion source may be selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof. The zinc ion source may be selected from the group consisting of zinc oxide, zinc sulfate, zinc chloride, zinc citrate, zinc lactate, zinc gluconate, zinc malate, zinc tartrate, zinc carbonate, zinc phosphate and a combination thereof.

In some embodiments, the tested oral care composition comprises arginine in free or salt form and a source of stannous ion or zinc ion. The stannous ion source may be selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof. In some embodiment, the stannous ion source is selected from stannous fluoride, stannous chloride and a combination thereof. The zinc ion source may be selected from the group consisting of zinc oxide, zinc sulfate, zinc chloride, zinc citrate, zinc lactate, zinc gluconate, zinc malate, zinc tartrate, zinc carbonate, zinc phosphate and a combination thereof. In some embodiment, the zinc ion source is selected from zinc citrate, zinc oxide, zinc lactate, zinc chloride and a combination thereof. In some embodiments, the zinc ion source is a combination of zinc citrate and zinc oxide. In some embodiments, the tested oral care composition comprises arginine in free or salt form, a source of stannous ion and a source of zinc ion. In some embodiments, the tested oral care composition comprises arginine in free or salt form and cetylpyridinium chloride (CPC).

The composition to be tested may be a solution containing a compound of interest. The compound of interest may be first diluted with or dissolved in a solution, e.g., water or a buffered solution, e.g., phosphate buffered saline (PBS). Then, the biofilm-coated object is treated with the compound by placing the biofilm-coated object in the solution containing the test compound. The tested compound may be an anti-microbial agent or a basic amino acid in free or salt form, e.g., arginine in free or salt form.

After the biofilm-coated object is treated with the composition of interest, the mechanical properties of a biofilm are measured by the spinning-disc rheometry analysis as disclosed in this disclosure, e.g., any of Methods 1 et seq.

The biofilm detachment efficacy of tested compositions may be determined by comparing one or more parameters of the test composition selected from angular velocity (ω) at which the first reduction in torque occurs, shear stress (τ) at which the first reduction in torque occurs, biofilm momentum coefficient (C_{B}), area under the curve (AUC) of the torque-angular velocity curve, and a combination thereof with those of a control composition. The control composition may be a composition, e.g., an oral care composition, e.g., toothpaste, tooth gel, or mouthwash, or a solution, e.g., water or a buffered solution, e.g., phosphate buffered saline (PBS), that does not contain any ingredient that may affect mechanical properties of a biofilm, e.g., anti-microbial agents. In some embodiments, the control composition is a solution, e.g., water or a buffered solution, e.g., phosphate buffered saline (PBS). In some embodiments, the control composition is the same as the tested composition except that the control composition does not contain any ingredient that may affect mechanical properties of a biofilm. For example, if the tested composition is a toothpaste containing arginine, the control composition may be a toothpaste containing no ingredient such as arginine that may affect mechanical properties of a biofilm. In some embodiments, if the tested composition is a solution, e.g., water or a buffered solution, e.g., phosphate buffered saline (PBS), containing a compound of interest, the control composition is the same as the tested solution except that the control composition does not contain the compound.

In another aspect, the invention provides a method of reducing or removing biofilm, e.g., dental plaque, from teeth, comprising an effective amount of an oral care composition to the oral cavity of a subject in need, wherein the oral care composition is identified by a method of identifying a composition having biofilm detachment activity disclosed in this disclosure, e.g., any of Methods 2 et seq.

### EXAMPLES

Spinning-disc rheology was adapted to analyze biofilm detachment from surfaces. S. gordonii biofilms were grown on 3D printed coupons for 5 days. The model for the coupons was designed in SolidWorks (Dassault Systèmes). Coupons were 3D printed using a Prime 30 PolyJet 3D printer (Objet, Stratasys) using RGD720 photopolymer for the printing material (Stratasys). The coupon was printed at a resolution of 0.0008 inches. The coupon surface was sanded used P300 sandpaper to create a rougher surface for bacteria to attach. Prior to inoculating, coupons were sterilized in 70% ethanol.

S. gordonii wild type strain DL1 was used in this study. Overnight cultures were prepared by inoculating 10 mL of brain heart infusion broth (Oxoid; BHI) with a colony of S. gordonii and incubated statically overnight at 37° C. with 5% CO2. Sterile 40 mm coupons were placed in a Petri dish containing 40 mL BHI, supplemented with 0.5% sucrose. Coupons were inoculated with 400 μL of overnight culture. Biofilms were incubated in a humidified chamber at 37° C. with 5% CO2, on an orbital shaker at 150 rpm. Every 24 h the media was replenished. Biofilms were grown for 5 days.

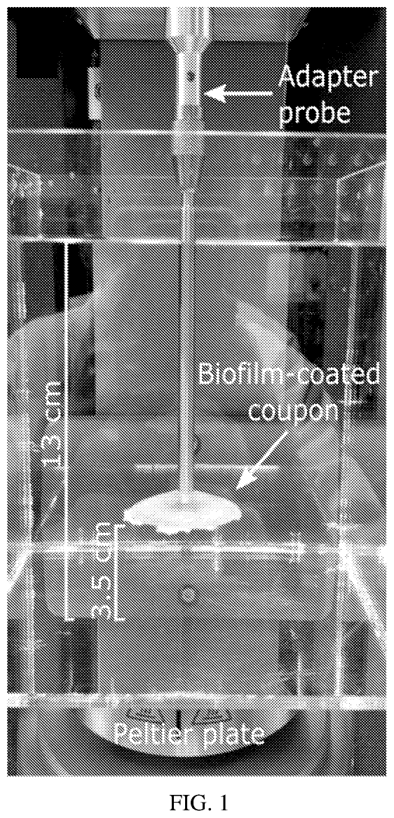

Biofilms were analyzed on a Discovery Hybrid Rheometer-2 (HD-2) (TA Instruments). A 15×15 cm square clear acrylic container filled with 2.8 L reverse osmosis water was transferred onto the Peltier plate. Biofilm-coated coupons were immersed and attached to the rheometer shaft using a custom-made adapter probe. The gap distance between the bottom of the container and the coupon was set to 3.5 cm (FIG. 1). Immersed coupons were spun at an angular velocity (ω) range of 0.1-300 rad·s⁻¹, incrementing the speed over 360 seconds, and the resulting torque (T), a measurement of resistance to rotation, was measured across this velocity range.

Data was collected using TRIOS v5 software (TA instruments), with raw data exported in excel. Data was transformed, and calculations performed in excel. Data was visualized and statistical analysis performed in GraphPad Prism v8 (GraphPad Software). All statistical comparisons were performed using a Student's t-test, with p<0.05 indicating significance.

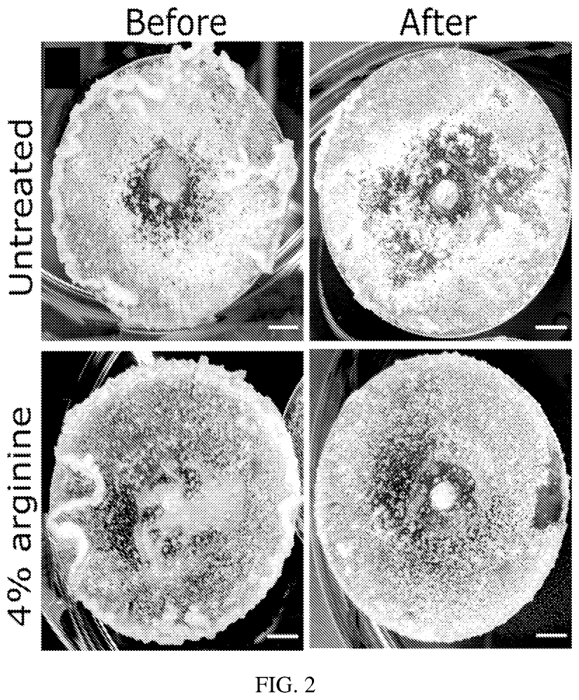

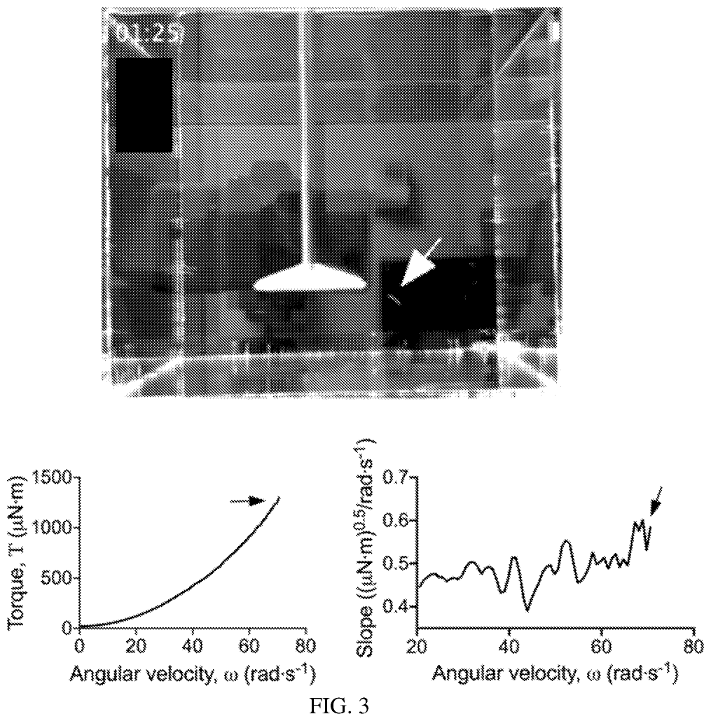

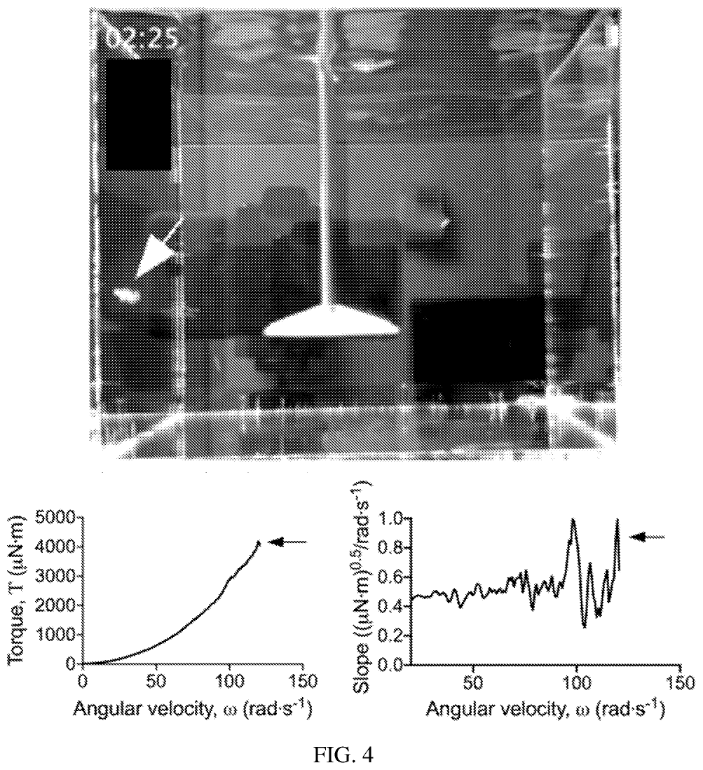

Detachment of biofilm aggregates was observed, particularly at the higher velocity regimes. These detachment events appeared to correlate to reductions in torque, with both small (FIG. 3) and larger (FIG. 4) aggregate detachments detected. After analysis there remained biofilm still attached to the coupon surface (FIG. 2). The remaining biofilm was not removed with repeated analysis.

To more easily observe the changes in torque associated with biofilm detachment, the torque-angular velocity data was linearized and transformed. The data was linearized by taking the square root of the torque. The running slope of 5 data points of the linearized data was determined. This transformed data was linearized after 20 rad·s⁻¹. Therefore, final transformed data is presented as the running slope of the linearized data against angular velocity, starting at 20 rad·s⁻¹. Using this transformed analysis, the reductions in torque were emphasized (FIGS. 3 and 4).

Having validated the sensitivity of the adapted spinning-disc rheometry, this assay was used to determine how arginine treatment influenced biofilm mechanics in regard to biofilm removal. Five day S. gordonii biofilms were treated with either PBS (untreated control) or 4% arginine for 2 min. This short treatment time was selected to mimic the time that a person would typically brush their teeth. After treatment, Biofilms were analyzed on a Discovery Hybrid Rheometer-2 (HD-2) (TA Instruments) as described above. Four biological replicates were analyzed, each with 2-4 technical replicates (total N=11).

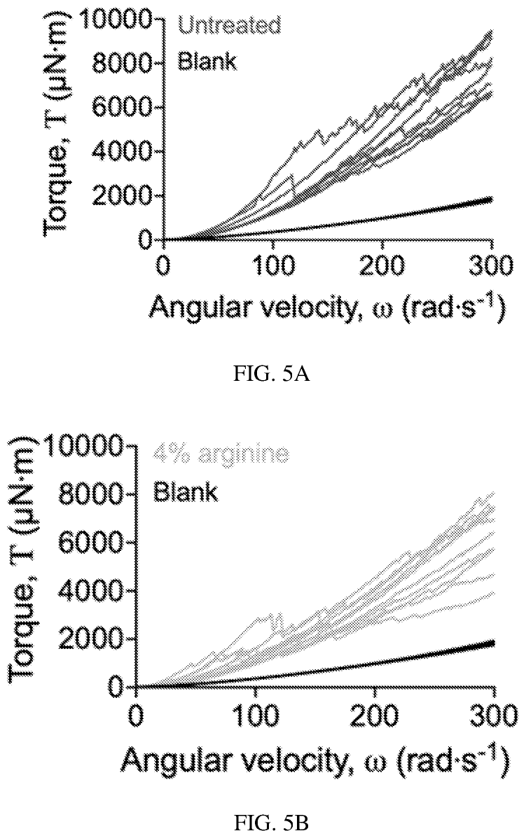

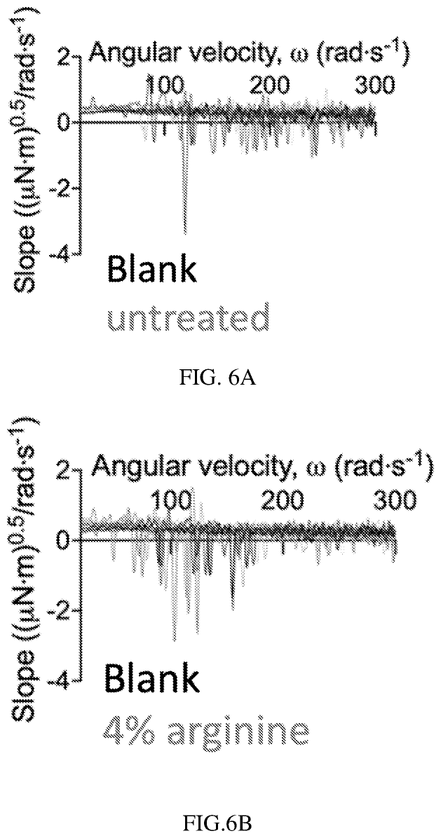

Macroscopically, arginine treatment did not appear to affect biofilm morphology, or the amount of remaining biofilm attached to the coupon after rheometry analysis (FIG. 2). However, arginine treated biofilms displayed reduced torque, compared to untreated biofilms (FIGS. 5A and B). This indicates that coupons with arginine treated biofilms could rotate more easily across the assayed angular velocity range. This is further highlighted by the transformed data, where greater changes in torque, indicated by more negative slope values, were observed for arginine treated biofilms, compared to untreated (FIGS. 6A and B). Both treated and untreated biofilms increased torque values compared to the coupon alone (FIGS. 5 and 6).

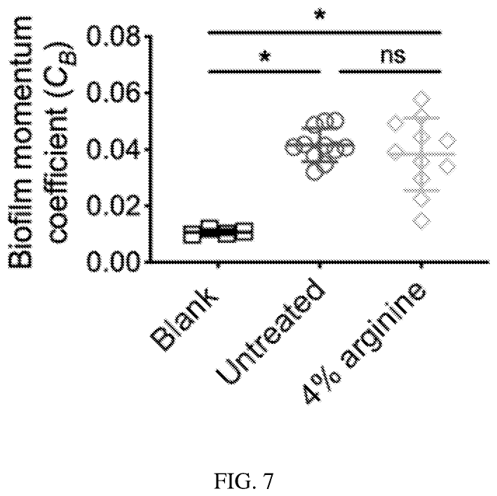

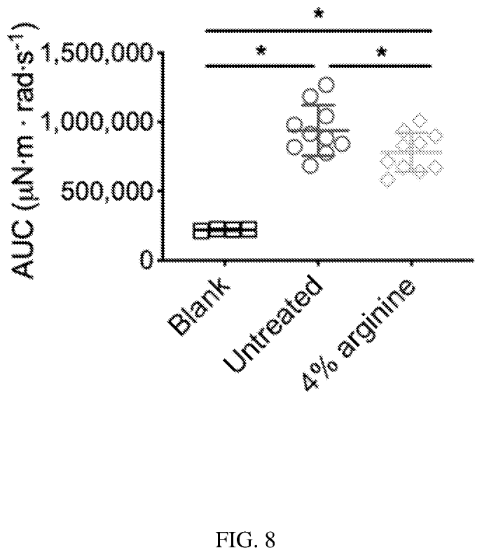

To quantify the mechanical differences between arginine treated and untreated S. gordonii biofilms, the biofilm momentum coefficient (C_{B}) across the turbulent regimes of 200-300 rad·s⁻¹ was determined according to equation 1:

$${C}_{B} = \frac{slope}{k} \qquad ( 1 )$$

where k is a constant for the system, defined by:

$$k = \frac{ρ \cdot {r}^{5}}{2} \qquad ( 2 )$$

where ρ is the density of the fluid, in this case water (997 kg/m³) and r is the radius of the coupon (0.02 m). The biofilm momentum coefficient is a dimensionless unit that is an indication of the drag caused by the biofilm, which in turn is related to the thickness and roughness of biofilm. Therefore, a higher coefficient is associated with more drag on the coupon, due to increased amount of adhered biofilm. Both untreated and treated S. gordonii biofilms had biofilm momentum coefficients significantly greater than the coupon alone (FIG. 7), indicating that the presence of attached biofilm significantly hampered rotation of the coupon. Although there was no statistical difference between the coefficient of untreated and treated biofilms, there was greater variability for arginine treated biofilms, as indicated by the standard deviation; 0.006 for untreated biofilms and 0.013 for arginine treated biofilms (FIG. 7). This indicates that there was greater heterogeneity of arginine treated biofilms across 200-300 rad·s⁻¹, compared to untreated biofilms. To look into these differences further, the AUC of the torque-angular velocity curves was determined (FIG. 5B). The area under the curve (AUC) of the torque-angular velocity curves was determined using the analysis function in GraphPad Prism. Unlike the biofilm momentum coefficient, which only takes into consideration coupon rotation between 200-300 rad·s⁻¹, AUC considers the rotation across the whole analyzed range. From this analysis, both untreated and treated S. gordonii biofilms had significantly greater AUC compared to the coupon alone, consistent with the fouling coefficient (FIG. 8). However, arginine treated biofilms had significantly reduced AUC, compared to untreated biofilms (FIG. 8). This suggests that, when also considering the lower velocity ranges, less work was required for rotation of the coupon coated with arginine treated biofilms, compared to untreated biofilms.

From the transformed analysis, it also appeared that for arginine treated biofilms, the changes in torque, associated with biofilm detachment events, occurred at lower angular velocity ranges, compared to untreated biofilms (FIGS. 6A and B). To quantify these differences, the angular velocity where the first reduction in torque occurred was converted to the shear stress acting on the outer edge of the coupon, according to equation 3.

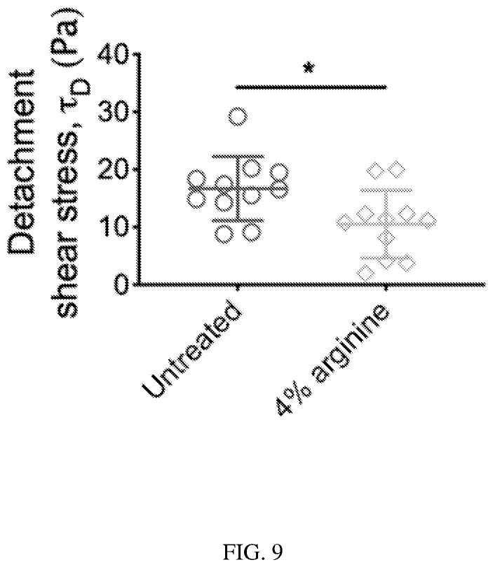

τ=\sqrt{τ_{φ}²+τᵣ²} (3)

where, τ_{φ} is the shear stress acting in the circumferential direction and τᵣ is the shear stress acting radially. This analysis revealed that reductions in torque occurred at significantly lower shear stresses for arginine treated biofilms, compared to untreated (FIG. 9). This indicates that arginine treated biofilms were detaching from coupons at lower shear stresses, suggesting that they were more easily removed by external shear forces, compared to untreated biofilms.

Next, the biofilm disrupting effect of arginine was compared with that of glycine and lysine by the spinning-disc rheometry assay to determine if biofilm disrupting effect is a general property of amino acids, or specific to arginine. Seven day S. gordonii biofilms were treated with either PBS (untreated control) or 4% arginine, or equal molar concentrations of glycine or lysine (0.23M) for 2 min. Glycine and lysine were selected as control amino acids. Arginine-treated biofilms displayed reduced torque, compared untreated biofilms. In contrast, glycine- and lysine-treated biofilms had torque-displacement profiles similar to untreated biofilms.

To quantify the mechanical differences between arginine-treated and untreated or glycine- and lysine-treated S. gordonii biofilms, the biofilm momentum coefficient (C_{B}) across the turbulent regimes of 200-300 rad·s⁻¹ was determined according to equation 1:

$${C}_{B} = \frac{slope}{k} \qquad ( 1 )$$

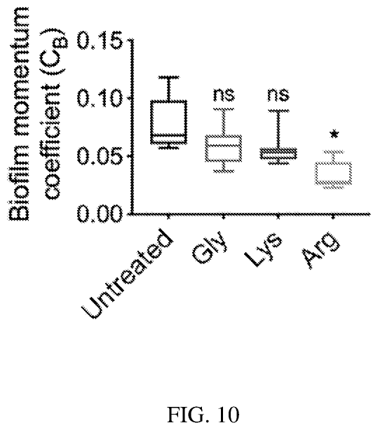

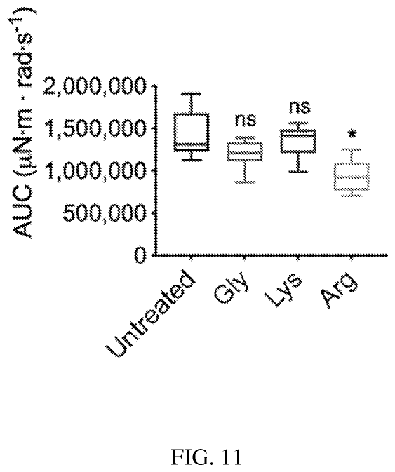

Glycine- and lysine-treated S. gordonii biofilms had biofilm momentum coefficients similar to untreated biofilms (FIG. 10). However, arginine-treated S. gordonii biofilms had a significantly lower biofilm momentum coefficient, compared to untreated biofilms (FIG. 10). This indicates that there was less drag caused by arginine-treated biofilms compared to either untreated or glycine- and lysine-treated biofilms. To look into these differences further, the area under the curve (AUC) of the torque-angular velocity curves was compared (FIG. 11). Unlike the biofilm momentum coefficient, which only takes into consideration coupon rotation between 200-300 rad·s⁻¹, AUC considers the rotation across the whole analyzed range. Consistent with the biofilm momentum coefficient analysis, there were no significant differences between the AUC of both untreated and glycine- and lysine-treated biofilms (FIG. 11). However, arginine-treated biofilms had significantly reduced AUC, compared to untreated biofilms (FIG. 11). This suggests that when also considering the lower velocity ranges, less work was required for rotation of the coupon of arginine-treated S. gordonii biofilms, compared to both untreated biofilms and glycine- and lysine-treated biofilms.

The angular velocity where the first reduction in torque occurred was converted to the shear stress acting on the outer edge of the coupon, according to equation 3.

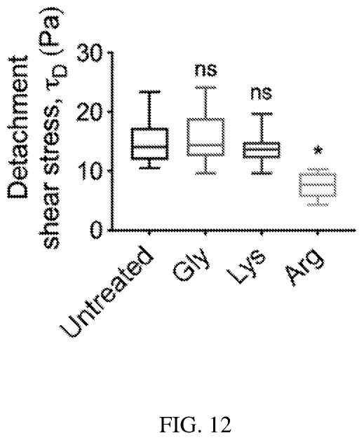

τ=\sqrt{τ_{φ}²+τᵣ²} (3)

where, τ_{φ} is the shear stress acting in the circumferential direction and τᵣ is the shear stress acting radially. This analysis revealed that there was no significant difference in the detachment shear stress of glycine- or lysine-treated S. gordonii biofilms compared to untreated. However, reductions in torque occurred at significantly lower shear stresses for arginine-treated biofilms, compared to untreated (FIG. 12). This indicates that arginine-treated biofilms were detaching from coupons at lower shear stresses, suggesting that they were more easily removed by external shear forces, compared to untreated or glycine- and lysine-treated S. gordonii biofilms.

This study shows that the spinning-disc rheometry assay is highly sensitive at detecting biofilm detachment and possible structural rearrangements with increasing shear forces. This methodology is also sensitive at detecting mechanical changes to the biofilm architecture that are not visually apparent.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. A method of monitoring the detachment of a biofilm from the surface of an object or rearrangement of the structure of the biofilm in response to shear stress; comprising:
a) providing a biofilm-coated object;
b) immersing the biofilm-coated object in a liquid;
c) spinning the biofilm-coated object with increasing angular velocity (ω), wherein the spinning generates shear stress;
d) monitoring torque (T) in step c) to obtain torque-angular velocity curve;
e) optionally, linearizing and transforming the torque-angular velocity curve into slope (torque^{1/2}/angular velocity)-angular velocity curve; and
f) determining one or more parameters selected from angular velocity (ω) at which the first reduction in torque occurs, shear stress (τ) at which the first reduction in torque occurs, biofilm momentum coefficient (C_{B}), area under the curve (AUC) of the torque-angular velocity curve, and a combination thereof.

2. The method of claim 1, wherein the object is a coupon, a tooth or a hydroxyapatite disk.

3. The method claim 1, wherein the biofilm is a biofilm of bacteria selected from S. sanguis, S. mutans, S. gordonii, S. parasangiiis, S. rattus, S. milieri, S. anginosus, S. faecalis, A. naeslundii, A. odonolyticus, L. cellobiosus, L. brevis, L. fermentum, P. gingivalis, T. denticola, and a combination thereof.

4. The method claim 1, wherein the biofilm is a biofilm of S. gordonii.

5. The method claim 1, wherein in step c), the biofilm-coated object is spun at an angular velocity (o) of from 0 to 300 rad·s⁻¹.

6. The method claim 1, wherein the angular velocity (ω) at which the first reduction in torque occurs represents the angular velocity (ω) at which the first detachment of biofilm from the object occurs and wherein the shear stress (τ) at which the first reduction in torque occurs represents the shear stress (τ) at which the first detachment of biofilm from the object occurs.

7. The method claim 1, wherein the biofilm momentum coefficient (C_{B}) is determined between 200-300 rad·s⁻¹.

8. A method of identifying compositions having biofilm detachment activity or evaluating the biofilm detachment efficacy of compositions; comprising:
a) providing a biofilm-coated object;
b) treating the biofilm-coated object with a composition;
c) immersing the biofilm-coated object in a liquid;
d) spinning the biofilm-coated object with increasing angular velocity (ω), wherein the spinning generates shear stress;
e) monitoring torque (T) in step c) to obtain torque-angular velocity curve;
f) optionally, linearizing and transforming the torque-angular velocity curve into slope (torque^{1/2}/angular velocity)-angular velocity curve
g) determining one or more parameters selected from angular velocity (ω) at which the first reduction in torque occurs, shear stress (τ) at which the first reduction in torque occurs, biofilm momentum coefficient (C_{B}), area under the curve (AUC) of the—angular velocity curve, and a combination thereof, and
h) comparing the one or more parameters of the composition with those of a control composition.

9. The method of claim 8, wherein the object is a coupon, a tooth or a hydroxyapatite disk.

10. The method of claim 8, wherein the biofilm is a biofilm of bacteria selected from S. sanguis, S. mutans, S. gordonii, S. parasangiiis, S. rattus, S. milieri, S. anginosus, S. faecalis, A. naeslundii, A. odonolyticus, L. cellobiosus, L. brevis, L. fermentum, P. gingivalis, T. denticola, and a combination thereof.

11. The method of claim 8, wherein the biofilm is a biofilm of S. gordonii.

12. The method of claim 8, wherein the composition is an oral care composition or a solution comprising an anti-microbial agent or a basic amino acid in free or salt form, e.g., arginine in free or salt form.

13. The method of claim 8, wherein the composition comprises arginine in free or salt form.

14. The method of claim 13, wherein the composition further comprise an anti-microbial agent selected from a stannous ion source, a zinc ion source and cetylpyridinium chloride (CPC).

15. The method of claim 8, wherein in step b), the biofilm-coated object is treated with the composition for 1-3 minutes or about 2 minutes.

16. The method of claim 8, wherein in step c), the biofilm-coated object is spun at an angular velocity (ω) of from 0 to 300 rad·s⁻¹.

17. The method of claim 8, wherein the angular velocity (ω) at which the first reduction in torque occurs represents the angular velocity (ω) at which the first detachment of biofilm from the object occurs and wherein the shear stress (τ) at which the first reduction in torque occurs represents the shear stress (τ) at which the first detachment of biofilm from the object occurs.

18. The method of claim 8, wherein reduction in the one or more parameters compared to the control composition indicates that the compound has biofilm detachment activity.

19. The method of claim 8, wherein the biofilm momentum coefficient (C_{B}) is determined between 200-300 rad·s⁻¹.

20. A method of reducing or removing dental plaque from teeth, comprising an effective amount of an oral care composition to the oral cavity of a subject in need, wherein the oral care composition is identified by a method of claim 8.
